(19) United States
(12) Patent Application Publication
(10) Pub. No.: US 2006/0148068 A1
(43) Pub. Date: Jul. 6, 2006

(54) Biological sensor

(76) Inventor: Jacques Noury, Paris (FR)

Correspondence Address: STEPTOE & JOHNSON LLP, 1330 CONNECTICUT AVENUE, N.W., WASHINGTON, DC, 20036, US

(21) Appl. No.: 10/541,617

(22) PCT Filed: Jan. 8, 2004
(86) PCT No.: PCT/FR04/00024

§ 371 Date: Jul. 7, 2005

(30) Foreign Application Priority Data
Jan. 9, 2003 (FR) 03/00195

(51) Int. Cl. C12M 1/34

(52) U.S. Cl. 435/288.1; 435/287.3; 435/288.7; 435/287.6; 435/309.1

(57) ABSTRACT

The invention relates to a portable and autonomous biological detector 1 enabling the presence of biological agents such as the bacteria, viruses, protozoan, or toxin type to be detected in a sample.

In the same body 2, this detector integrates means to extract a sample from the environment, be it solid, liquid or gaseous; means 4 for the biological culture or magnification of said sample; detection means 5 inducing a reaction, for example on an impregnated strip 12, said reaction being either colourmetric and visible to the naked eye thanks to a transparent viewfinder 9, or detectable by a separate system. The sample extraction means 8 incorporate a plug 3 able to be screwed or nested onto the body 2 of the biological detector and a lip 15 ensuring its sealing and the culture medium 11 is contained in a breakable ampoule 10 so as to allow the sample to be brought into contact with said culture medium 11.

The technical scope of the present invention is that of devices enabling the presence of biological agents to be detected in samples taken from a suspicious environment.

Techniques enabling the presence of biological agents to be detected are well known in prior art. Up to now, the use of these techniques required unwieldy devices and had to be carried out in a laboratory to which the samples had to be sent for analysis.

This way of proceeding, although perfectly reliable and controlled, nevertheless causes delays which, according to the situation, may become dramatic.

The aim of the invention is to propose a detection device for biological agents that can be used on a small-sized sample in the field and which does not require the samples to be transferred for analysis. By small-sized, we mean a sample containing, for example, between 100 and 1,000 spores or a few nanograms.

The invention thus relates to a portable and autonomous biological detector enabling the presence of biological agents of the bacteria, viruses, protozoan, or toxin type to be detected, wherein it integrates into a same body:
means to extract a sample from the environment, be it solid, liquid or gaseous,
means for the biological culture or magnification of said sample,
detection means inducing a reaction, said reaction being either colourmetric and visible to the naked eye, or detectable by a separate system.

According to one characteristic of the invention, the detection reaction of the device is detected by a physical and/or optical system such as a laser or by infrared light, ultraviolet light or by electron beam.

According to another characteristic of the invention, the sample extraction means are of the manual or automatic type.

According to one embodiment of the invention, the sample extraction means are in the form of a sampling brush.

According to another embodiment of the invention, the sample extraction means incorporate a biocollector.

According to yet another embodiment of the invention, the sample extraction means are constituted by a syringe.

According to one characteristic of the invention, the sample extraction means are in the form of a plug able to be screwed or nested onto the body of the biological detector and incorporating a lip ensuring its sealing with this body, such plug being made of stainless metal or a plastic metal and provided with the instrument enabling the extraction of the samples.

According to another characteristic of the invention, the culture or magnification means incorporate a culture or reaction medium contained in a breakable ampoule so as to allow the sample to be brought into contact with said culture medium.

According to another characteristic of the invention, the magnification means for the samples comprise a culture or chemical reaction chamber containing a culture or magnification medium adapted to the type of suspected biological agent, said chamber being provided with heating means.

According to one characteristic of the invention, the means to detect biological agents comprise biological substances such as enzymes, antibodies, proteins, cellular fragments or sequences of DNA or RNA.

According to another characteristic of the invention, the biological substances are associated with chemical substances such as metalloids, colloids, or colorants whose reaction with an antigen enables the visualisation of the detection of the suspected biological agent.

According to a particular embodiment of the invention, the means to detect biological agents comprise a support impregnated with specific antibodies for the suspected biological agent, enabling the immuno-detection of said biological agent.

According to one characteristic of the invention, the device incorporates a septum placed near to the culture chamber so as to enable the extraction by syringe of said culture.

According to one characteristic of the invention, the detection target may be the suspected biological agent, a product of its metabolism, a molecule or its metabolites.

According to a particular embodiment of the invention, the suspected biological agent is anthrax (Bacillus anthracis) or the smallpox virus.

According to another characteristic of the invention, the detector is in the form of a tube incorporating at one end means to extract the sample, in its median part the means enabling the culture or magnification of said sample and at the other end the detection means of the suspected biological agent, these means being associated with sealing means.

According to yet another characteristic of the invention, the detector constitutes packaging means for the magnified culture enabling its subsequent analysis and use as evidence.

According to one embodiment of the invention, the biological detector incorporates a system of power supply that supplies the heating means.

According to one embodiment of the invention, the biological detector comprises a pilot light indicating the end of the culture or biological magnification phase and the onset of the detection phase.

According to one characteristic of the invention, the detector comprises security means preventing it from being opened, deliberately or not, after the sample has been inserted.

The invention also relates to the application of the detector to the simultaneous detection of several biological agents.

One advantage of the biological detector according to the invention lies in that it enables the analysis of a suspicious environment on the spot and within a short space of time.

Another advantage of the detector according to the invention lies in its simplicity of use, making it able to be used by both specialists and lay people.

Another advantage of the biological detector according to the invention lies in that it provides maximum safety to the user thanks to its total tightness once the sample has been inserted.

Another advantage of the biological detector according to the invention lies in the rapidity of the diagnostic.

Other advantages, particulars and characteristics of the invention will become more apparent from the additional description given hereafter by way of illustration and with reference to the appended drawings, in which:

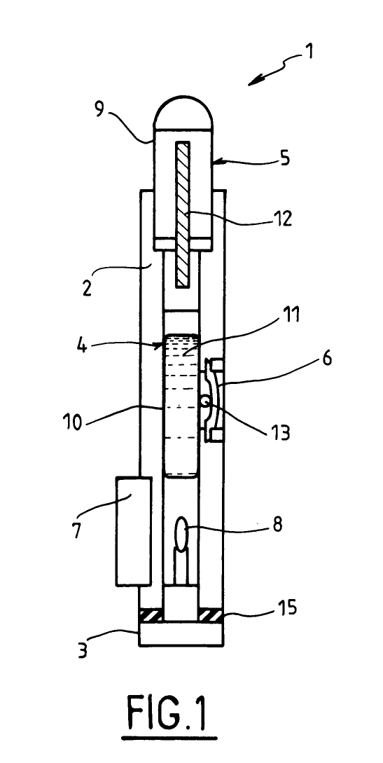

FIG. 1 shows the biological detector according to the invention, before use,

FIG. 2 shows means to extract a sample during use,

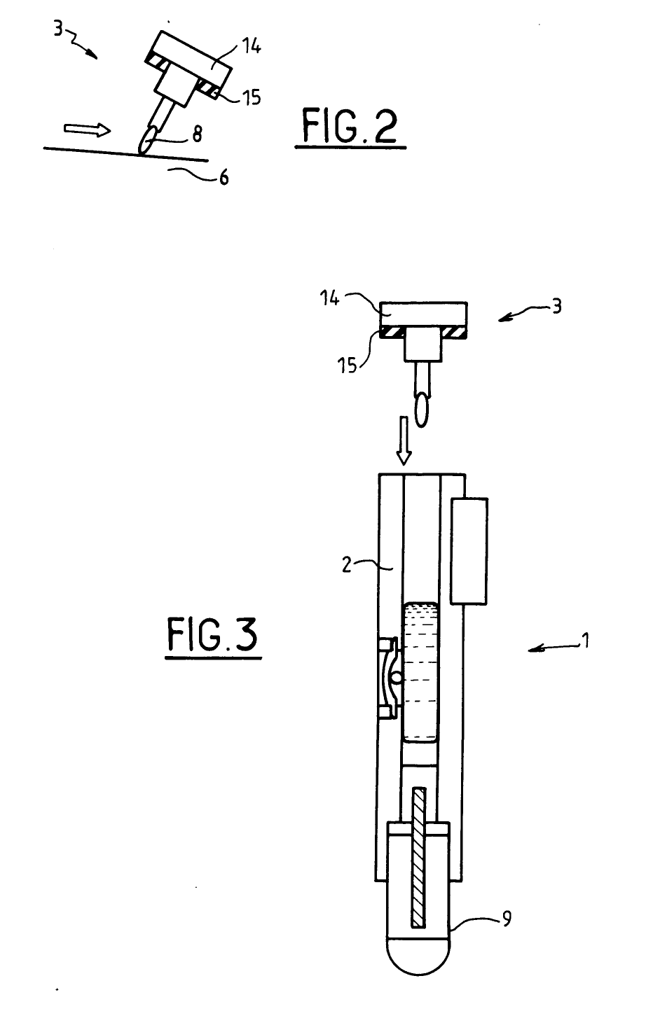

FIG. 3 shows the insertion of the sample extraction means into the detector,

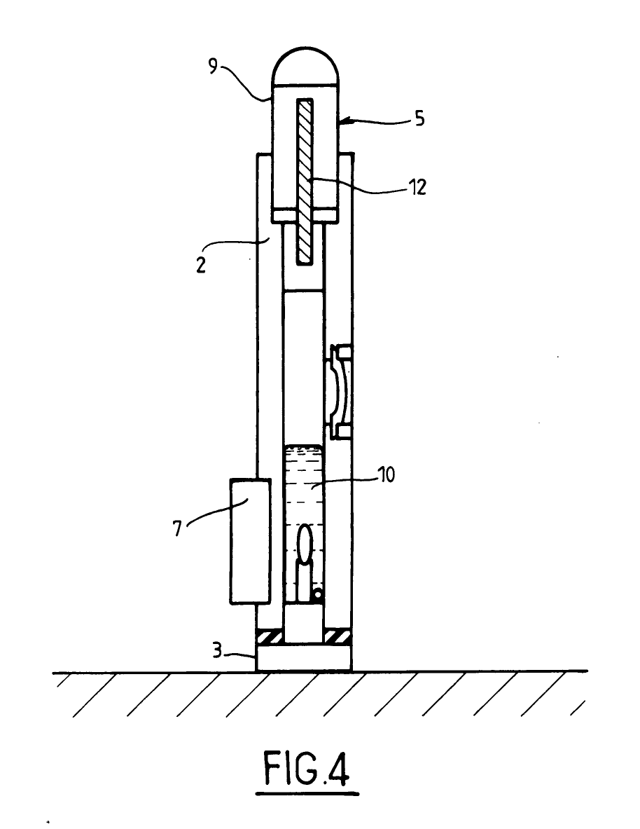

FIG. 4 shows the sample magnification stage, and

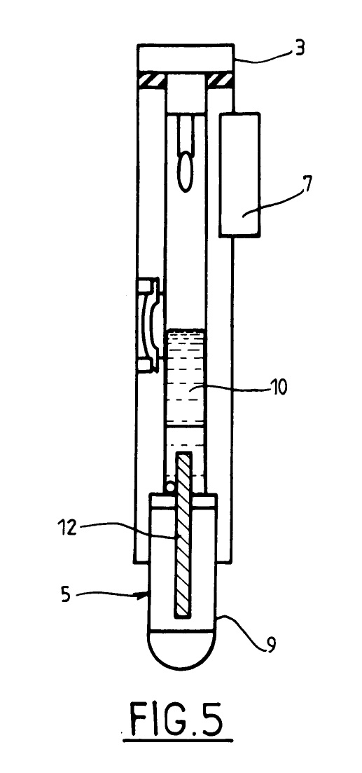

FIG. 5 shows the detection stage of the suspected biological agent.

FIG. 1 shows the biological detector 1 according to the invention, constituted by a tubular body 2 comprising a plug 3 carrying the sample extraction means 8, sample culture means 4, constituted by an ampoule 10 containing a culture medium 11, and detection means 5 constituted by an impregnated strip 12 and having a viewfinder 9 permitting the reaction taking place on the strip 12 to be viewed. The reaction may occur on the strip by immuno-reaction. As many specific antibodies may be fixed to the strip 12 as biological agents to be detected. The detection target may be the suspected biological agent, a product of its metabolism, a molecule or its metabolites, for example a toxin secreted in the culture medium, a membrane antigen expressed in certain conditions, a specific enzyme or the vegetative form of a micro-organism in its encapsulated or spore-forming form, etc.

The sample extraction means are here of the manual type but they may also be of the automatic type and may incorporate a bio-collector. This may be in the form of a syringe.

The detection means may be an immuno-chromatography of the strip, such type being well known to the Expert. The detection means 5 for the biological agents comprise biological substances such as enzymes, antibodies, proteins, cellular fragments or DNA or RNA sequences.

The body 2 of the biological detector 1 also has, in the culture medium 4 part, a septum 6 which enables samples to be taken. At this part there are also means to break the ampoule 10, shown here in the form of a ball 13.

The detector 1 also comprises heating means 7 integral with the body 2 whose temperature can be modulated using a modulator. An power supply system supplying the heating system 7 may be provided.

The biological substances are associated with chemical substances such as metalloids, colloids or colorants whose reaction with the antigen enables a visualisation of the detection of the suspected biological agent.

The reaction may be detected using a physical and/or optical system such as a laser or by infrared, ultraviolet light or electron beam.

FIG. 2 shows the plug 3. This plug 3 carries the sample extraction means 8 shown in this example by a sampling brush. The plug 3 is constituted by a washer 14 of stainless metal or plastic material and a seal 15. A hermetic system is thus obtained. The sample extraction means 8 enable samples to be taken from an environment 16 which may be the ambient air or a liquid or solid environment.

FIG. 3 shows the plug 3 inserted into the tubular body 2 of the detector 1. As may be seen, it is the washer 14 that closes the body 2 and the seal 15 ensures that the plug 3/body 2 assembly is hermetic.

FIG. 4 shows the biological detector 1 after the sample has been inserted and the ampoule 10 broken during the biological magnification phase. During the magnification phase of the sample, the detector 1 is placed in a vertical position on the plug 3. This arrangement enables the liquid culture medium to be slid to the bottom of the detector 1 to come into contact with the sample taken using the sampling brush 8 and the heating means 7, this is in order to allow the optimal development of the biological agents present in the sample.

Note that detection does not require extended contact of the culture medium with the detection means since a few seconds is enough. The device is returned to the position shown in FIG. 4 and the reaction is left to develop. The reaction time may vary according to the detection system and according to the parameters peculiar to each agent to be detected.

FIG. 5 shows the biological detector 1 after biological magnification of the sample at the onset of the detection phase. The detector is tipped into the vertical position opposed to the previous one so as to bring the biological culture into contact with the detection means 5. The detection reaction may be seen by the operator through the viewfinder 9. This detector may thus constitute packaging means for the magnified culture for its subsequent analysis and use as evidence.

FIGS. 2 to 5 illustrate the biological agent detection process using the biological detector 1 according to the invention. This process, which will now be described, incorporates four stages, each illustrated by one of the Figures:

FIG. 2 illustrates the first stage, the extraction of a sample,

FIG. 3 illustrates the second stage, the insertion of the sample into the detector,

FIG. 4 illustrates the third stage, the biological culture or magnification of the sample, and

FIG. 5 illustrates the fourth stage, the detection strictly speaking.

The extraction of a sample is made using the plug 3 which carries the sampling means 8. Once the sample has been taken, the plug 3 is inserted into the tubular body 2 of the detector 1 and is secured by means (not shown) that prevent it being reopened. This plug 3 may be a screw plug that is tamperproof once screwed on.

Then the detector is turned upside down and placed on the plug, the ampoule 10 being broken using breaking means 12 thereby bringing the sample into contact with the culture medium 11. This stage, the culturing, may last 5 to 90 minutes, depending on the suspected biological agent, the culture medium and the sampling medium. The heating means 7 enable the temperature conditions adapted to the suspected biological agent to be obtained.

Once the culture phase is finished, which may be signalled by a pilot light set at the required culture time (not shown), the detector 1 is once again inverted so as to bring the culture medium into contact with the detection means 5, here constituted by an impregnated strip 12. The device is brought back into the position shown in FIG. 4 and after the reaction time, which varies according to the agent, the transparent part 9 of the tube enables any detection reaction to be seen.

The culture medium or the medium enabling a biochemical magnification reaction is well known to the Expert. By way of example, the medium named LB can be mentioned in this domain. The culture medium is naturally different according to the suspected biological agent. It may be adapted for the culture of one or several agents simultaneously. A classical liquid culture medium well known to the Expert is aerobic or anaerobic when bacteria are suspected, a living cell culture when a virus is suspected. The virus is obtained in the form of a biological sample (urine, blood, faeces).

Detection can take place with a magnification phase but by way of a variant it is possible for a frozen ampoule containing living cells to be used that will be inserted into the detector at the required moment.

In the example where the detector is applied to the detection of anthrax, the culture medium is that classically used for bacteria: viandox and distilled water. The detection means are thus constituted by an antibody/colloidal assembly which reacts with the specific antigens selected for the anthrax during an immunodetection reaction that is well known to the Expert and is currently employed. The medium known under the designation LB is currently used for the culture of anthrax.

The use of a plug of stainless metal or plastic material is envisaged. The plug may naturally be made of any material, however.

It is naturally possible for this detector to be used to identify other biological agents without departing from the scope of the invention.

1. A portable and autonomous biological detector (1) enabling the presence of biological agents of the bacteria, viruses, protozoan, or toxin type to be detected in a sample, wherein it integrates into a same body (2):
means (8) to extract a sample from the environment, be it solid, liquid or gaseous,
means (4) for the biological culture or magnification of said sample,
detection means (5) inducing a reaction, said reaction being either colourmetric and visible to the naked eye thanks to a transparent viewfinder (9), or detectable by a separate system.

2. A portable and autonomous biological detector (1) according to claim 1, wherein the reaction is detected by a physical and/or optical system such as a laser or by infrared light, ultraviolet light or by electron beam.

3. A biological detector (1) according to claim 1, wherein the sample extraction means (8) are of the manual or automatic type.

4. A biological detector (1) wherein the sample extraction means (8) are in the form of a sampling brush.

5. A biological detector (1) according to claim 1, wherein the sample extraction means (8) incorporate a biocollector.

6. A biological detector (1) according to claim 1, wherein the sample extraction means (8) are constituted by a syringe.

7. A biological detector according to claim 1, wherein the sample extraction means (8) are in the form of a plug (3) able to be screwed or nested onto the body (2) of the biological detector and incorporating a lip (15) ensuring its sealing with this body, such plug being made of stainless metal or a plastic metal and provided with the instrument enabling the extraction of the samples.

8. A biological detector according to claim 1, wherein the culture or magnification means incorporate a culture medium (11) contained in a breakable ampoule (10) so as to allow the sample to be brought into contact with said culture medium (11).

9. A biological detector according to claim 1, wherein the magnification means (4) for the samples comprise a culture chamber (10) containing a culture or magnification medium (11) adapted to the type of suspected biological agent, said chamber being provided with heating means (7).

10. A biological detector according to claim 1, wherein the means (5) to detect biological agents comprise biological substances such as enzymes, antibodies, proteins, cellular fragments or sequences of DNA or RNA.

11. A biological detector according to claim 10, wherein the biological substances are associated with chemical substances such as metalloids, colloids, or colorants whose reaction with an antigen enables the visualisation of the detection of the suspected biological agent.

12. A biological detector according to claim 1, wherein the means (5) to detect biological agents comprise a support impregnated with specific antibodies for the suspected biological agent, enabling the immuno-detection of said biological agent.

13. A biological detector according to claim 1, wherein it incorporates a septum (6) placed near to the culture chamber (10) so as to enable the extraction by syringe of said culture.

14. A biological detector according to claim 1, wherein the detection target may be the suspected biological agent, a product of its metabolism, a molecule or its metabolites.

15. A biological detector according to claim 1, wherein the suspected biological agent is anthrax (Bacillus anthracis) or the smallpox virus.

16. A biological detector according to claim 1, 15, wherein it is in the form of a tube (2) incorporating at one end means (8) to extract the sample, in its median part the means (4) enabling the culture or magnification of said sample and at the other end the means (5) to detect the suspected biological agent, these means being associated with sealing means.

17. A biological detector according to claim 1, wherein it constitutes packaging means for the magnified culture enabling its subsequent analysis and use as evidence.

18. A biological detector according to claim 1, wherein it incorporates a system of power supply that supplies the heating means (7).

19. A biological detector according to claim 1, wherein it comprises a pilot light indicating the end of the culture or biological magnification phase and the onset of the detection phase.

20. A biological detector according to claim 1, wherein it comprises security means preventing it from being opened, deliberately or not, after the sample has been inserted.

21. (canceled)
